(19) United States
(12) Patent Application Publication
(10) Pub. No.: US 2024/0368090 A1
(43) Pub. Date: Nov. 7, 2024

(54) SOLID STATE FORMS OF DAPRODUSTAT AND PROCESS FOR THEIR PREPARATION

(71) Applicant: REDDY’S LABORATORIES LIMITED, Hyderabad (IN)

(72) Inventors: BARLA RAJU, Ramagundam (IN); SHARMISTHA PAL, Bangalore (IN); SRINIVAS ACHANTA, Hyderabad (IN); DEBJIT BASU, Hyderabad (IN); PAVANI SANKAR REDDY, Hyderabad (IN); ARUN AEDHUNURI, Hyderabad (IN); SRINIVASULU RANGINENI, Hyderabad (IN); NAGA SAI SWAROOP GANNAVARAPU, Hyderabad (IN)

(21) Appl. No.: 18/654,436

(22) Filed: May 3, 2024

(30) Foreign Application Priority Data
May 5, 2023 (IN) 202341032042
May 26, 2023 (IN) 202341036415
Jul. 3, 2023 (IN) 202341044421
Jul. 28, 2023 (IN) 202341050927

(51) Int. Cl. C07D 239/62; A61K 31/515

(52) U.S. Cl. CPC C07D 239/62; A61K 31/515

(57) ABSTRACT

A crystalline form DP-9 of Daprodustat is provided, where an X-ray powder diffraction pattern of the crystalline form DP-9 shows one or more characteristic peaks at 6.4, 7.2, 7.5, 12.3, 15.1, 15.5, 16.6, 18.5, 19.3, 21.0, and 27.6±0.2 2θ.

### CROSS-REFERENCE TO RELATED APPLICATIONS

The present disclosure claims priorities to Indian provisional patent application No.: IN202341032042 filed on May 5, 2023, to Indian provisional application No.: IN202341036415 filed on May 26, 2023, to Indian provisional patent application No.: IN202341044421 filed on Jul. 3, 2023, and to Indian provisional patent application No.: IN202341050927 filed on Jul. 28, 2023, the contents of all of which are incorporated herein by reference in their entireties.

### TECHNICAL FIELD

The present disclosure relates to solid state forms and novel process for the preparation of Daprodustat.

### BACKGROUND

Aspects of the present disclosure relate to the novel process for preparing Daprodustat and their novel intermediates thereof.

The drug compound having the adopted name “Daprodustat” has chemical name: N-[(1,3-Dicyclohexylhexahydro-2,4,6-trioxopyrimidin-5-yl)carbonyl]glycine as below.

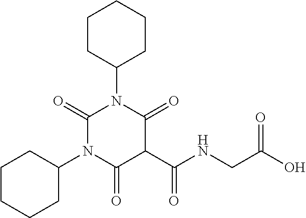 [SMILES: O=C(O)CNC(=O)C1C(=O)N(C2CCCCC2)C(=O)N(C2CCCCC2)C1=O]

Daprodustat is a hypoxia-inducible factor prolyl hydroxylase (HIF PH) inhibitor developed by Glaxo Smith Kline as JESDUVROQ oral tablets indicated for the treatment of anemia due to chronic kidney disease.

The compound Daprodustat is first described in PCT publication WO2007150011 and its use thereof for the treatment of anemia associated with renal disease is also disclosed. This literature describes two examples for the preparation of Daprodustat.

PCT publication WO2019052133 discloses two crystalline forms i.e. Form CS1 and Form CS9 of Daprodustat and its use in pharmaceutical composition.

Another PCT publication WO2020102302 describes crystalline Form 1, Form 2, Form 3 and Form 4 of Daprodustat. Their process of preparation and pharmaceutical composition is also disclosed in the present disclosure.

Another PCT publication WO2021031102 discloses crystalline form M and Form K of Daprodustat, its process of preparation and pharmaceutical composition.

PCT publication WO2023006986 discloses a one pot method for the preparation of Daprodustat.

PCT publication WO2024022998 discloses a process of preparing Daprodustat and crystalline Form H, Form D, Form C, Form G, Form F, Form B, Form E, Form A, Form N2, Form N4, Form K1 and Form K2 solid state forms.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, e.g., a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Daprodustat.

There is also a need in the art for the novel and improved method for synthesizing Daprodustat in commercially viable and cost-effective manner, which is suitable for large scale cGMP production for its pharmaceutical formulation manufacturing.

### SUMMARY

In one aspect, the present disclosure provides solid state forms of Daprodustat. In certain embodiment(s), a crystalline form DP-9 of Daprodustat is provided, where an X-ray powder diffraction pattern of the crystalline form DP-9 shows one or more characteristic peaks at 6.4, 7.2, 7.5, 12.3, 15.1, 15.5, 16.6, 18.5, 19.3, 21.0, and 27.6±0.2 2θ.

In another aspect, the present disclosure provides a pharmaceutical composition, comprising the crystalline form DP-9 of claim 1, and at least one pharmaceutically acceptable excipient.

In another aspect, the present disclosure provides a process for the preparation of solid-state forms of Daprodustat. In certain embodiment(s), the process includes: dissolving Daprodustat in polar aprotic solvent to obtain a reaction mixture; heating the reaction mixture to get a solution; optionally adding another organic solvent; isolating a solid obtained; drying in a suitable temperature; and optionally milling.

In another aspect, the present disclosure provides a process for preparing Daprodustat, where the process includes: converting compound 2 to compound 3 in presence of carbonyl reagent (C═O reagent) and amine reagent; and converting compound 3 to Daprodustat.

In another aspect, the present disclosure provides a process for preparing Daprodustat, wherein the process includes: converting compound 2 to compound 3 in presence of carbonyldiimidazole and Glycine methylester; and converting compound 3 to Daprodustat.

In another aspect, the present disclosure provides the novel process for preparing Daprodustat.

In another aspect, the present disclosure provides the novel process for preparing Daprodustat comprising intermediate of formula 2, formula 3 and further conversion into Daprodustat.

### BRIEF DESCRIPTION OF DRAWINGS

FIG. 1 is an illustrative X-ray powder diffraction pattern of Daprodustat ASD with Copovidone (1:3), prepared by the method of Example 1.

FIG. 2 is an illustrative X-ray powder diffraction pattern of Daprodustat ASD with HPMC (1:3), prepared by the method of Example 2.

FIG. 3 is an illustrative X-ray powder diffraction pattern of Crystalline Form DP-1 of Daprodustat, prepared by the method of Example 3.

FIG. 4 is an illustrative X-ray powder diffraction pattern of Crystalline Form DP-2 of Daprodustat, prepared by the method of Example 4.

FIG. 5 is an illustrative X-ray powder diffraction pattern of Crystalline Form DP-3 of Daprodustat, prepared by the method of Example 5.

FIG. 6 is an illustrative X-ray powder diffraction pattern of Crystalline Form DP-4 of Daprodustat, prepared by the method of Example 6.

FIG. 7 is an illustrative X-ray powder diffraction pattern of Crystalline Form DP-5 of Daprodustat, prepared by the method of Example 7.

FIG. 8 is an illustrative X-ray powder diffraction pattern of Crystalline Form DP-6 of Daprodustat, prepared by the method of Example 8.

FIG. 9 is an illustrative X-ray powder diffraction pattern of Crystalline Form DP-7 of Daprodustat, prepared by the method of Example 9.

FIG. 10 is an illustrative X-ray powder diffraction pattern of Crystalline Form DP-8 of Daprodustat, prepared by the method of Example 10.

FIG. 11 is an illustrative X-ray powder diffraction pattern of Crystalline Form DP-9 of Daprodustat, prepared by the method of Example 11.

FIG. 12 is an illustrative X-ray powder diffraction pattern of Daprodustat ASD with Eudragit (1:9), prepared by the method of Example 12.

FIG. 13 is an illustrative X-ray powder diffraction pattern of Daprodustat ASD with HPC (1:9), prepared by the method of Example 13.

FIG. 14 is an illustrative X-ray powder diffraction pattern of Daprodustat ASD with HPMC (1:9), prepared by the method of Example 14.

FIG. 15 is an illustrative X-ray powder diffraction pattern of Daprodustat ASD with HPMC-AS (1:9), prepared by the method of Example 15.

FIG. 16 is an illustrative X-ray powder diffraction pattern of Daprodustat ASD with PVPK-90 (1:9), prepared by the method of Example 16.

FIG. 17 is an illustrative X-ray powder diffraction pattern of Daprodustat ASD with Copovidone (1:9), prepared by the method of Example 17.

FIG. 18 is an illustrative X-ray powder diffraction pattern of Crystalline Form DP-10 of Daprodustat, prepared by the method of Example 18.

FIG. 19 is an illustrative X-ray powder diffraction pattern of Crystalline Form DP-11 of Daprodustat, prepared by the method of Example 19.

FIG. 20 is an illustrative X-ray powder diffraction pattern of Daprodustat with Nicotinamide (Form CC1), prepared by the method of Example 20.

FIG. 21 is an illustrative X-ray powder diffraction pattern of Daprodustat with Caffeine (Form CC2), prepared by the method of Example 21.

FIG. 22 is an illustrative X-ray powder diffraction pattern of Crystalline Form DP-9 of Daprodustat prepared by the method of Example 29.

FIG. 23 is DSC of Crystalline Form DP-9 of Daprodustat prepared by the method of Example 29.

FIG. 24 is TGA of Crystalline Form DP-9 of Daprodustat prepared by the method of Example 29.

FIG. 25 is an illustrative X-ray powder diffraction pattern of Crystalline Form of methyl (1,3-dicyclohexyl-6-hydroxy-2,4-dioxo-1,2,3,4-tetrahydropyrimidine-5-carbonyl)glycinate, prepared by the method of Example 27, method-2.

### DETAILED DESCRIPTION

In one aspect, the present disclosure relates to solid form of Daprodustat and the pharmaceutical compositions thereof.

In another aspect, the present disclosure provides crystalline Forms and amorphous solid dispersions (ASD's) of Daprodustat and their preparative processes.

In another aspect, the present disclosure provides crystalline forms of Daprodustat.

In another aspect, the present disclosure provides crystalline form DP-1 to DP-11 of Daprodustat.

In another aspect, the present disclosure provides crystalline form DP-9 of Daprodustat.

In another aspect, the present disclosure provides Crystalline form DP-9 of Daprodustat, characterized by PXRD pattern comprising the peaks at about: 6.4, 7.2, 7.5, 12.3, 15.1, 15.5, 16.6, 18.5, 19.3, 21.0 and 27.6±0.2 2θ.

In another aspect, the present disclosure relates to crystalline form DP-9 of Daprodustat, characterized by a PXRD pattern including the peaks at about: 6.38, 7.18, 7.52, 10.78, 12.25, 12.92, 15.36, 16.65, 17.18, 18.06, 18.67, 19.14, 19.72, 20.84, 22.73, 24.78, 26.94, 27.89±0.2° 2θ.

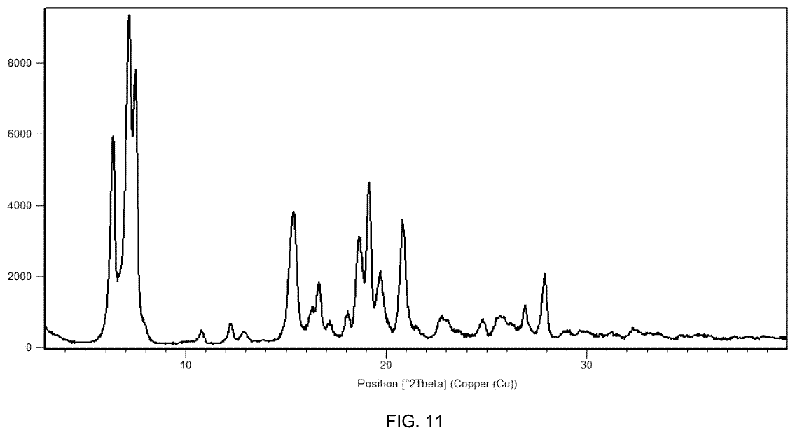

In another aspect, the present disclosure provides crystalline Form DP-9 of Daprodustat, characterized by a powder X-ray diffraction pattern, as illustrated by FIG. 11 and FIG. 22.

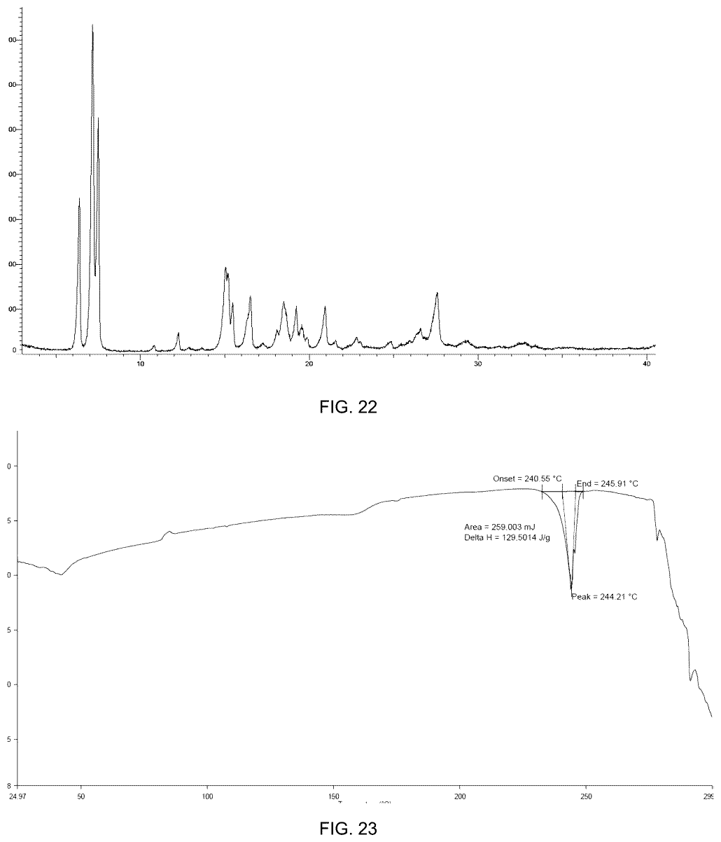

In another aspect, the present disclosure provides crystalline Form DP-9 of Daprodustat, characterized by DSC as shown in FIG. 23.

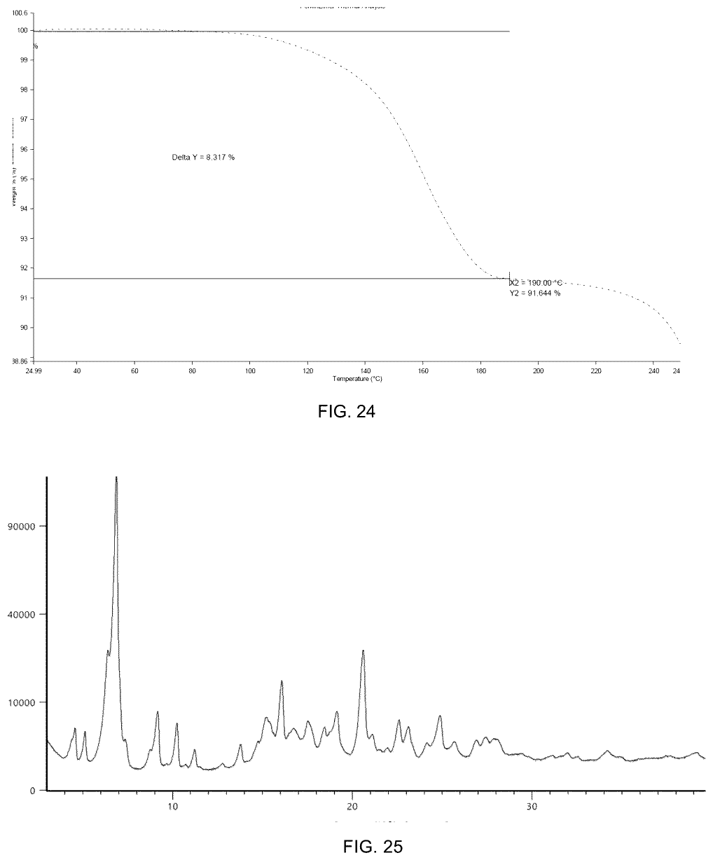

In another aspect of the present disclosure provides crystalline Form DP-9 of Daprodustat, characterized by TGA as shown in FIG. 24.

In another aspect, the present disclosure provides to crystalline form DP-9 of Daprodustat is DMF solvate.

In another aspect, the present disclosure provides crystalline form DP-9 of Daprodustat is Hemi-DMF solvate.

In another aspect, the present disclosure provides that the DMF content present in DP-9 is around 8.0 to 8.5% w/w determined by gas chromatography.

In another aspect, the present disclosure provides a general process for the preparation of crystalline Forms of Daprodustat, the process including,
dissolving Daprodustat in an organic solvent;
isolating the solid obtained; and
optionally drying.

In another aspect, the present disclosure provides a process for the preparation of crystalline Form DP-9 of Daprodustat, the process including:
dissolving Daprodustat in polar aprotic solvent, heating the reaction mixture to get a clear solution;
optionally adding another organic solvent;
isolating the solid obtained;
drying in a suitable temperature; and
optionally milling.

In another aspect, the present disclosure provides that the crystalline Form may be isolated by separating the solids from the solvent through suitable techniques known in the art such as evaporation, filtration, decantation and the like.

In another aspect of the present disclosure, the isolated solid may be dried under suitable drying conditions such as aerial drying, drying under vacuum or inert gas at a suitable temperature of about 25° C. or above.

In another aspect of the present disclosure, the crystalline forms of the present disclosure are stable under thermal, humid and stress conditions.

In another aspect of the present disclosure, the crystalline form DP-9 of Daprodustat which is physically & chemically stable under standard stability conditions like 25° C.-60% RH, 40° C.-75% RH and 2-8° C. for at least 3 months.

In another aspect, the present disclosure provides amorphous solid dispersions (ASD's) of Daprodustat.

In another aspect, the present disclosure provides an amorphous solid dispersion of Daprodustat with copovidone, HPMC, Eudragit, HPC, HPMC-AS, PVPK-90 and like.

In another aspect, the present disclosure provides crystalline salts of Daprodustat with Nicotinamide, Caffeine, L-Proline, aspartame and like.

In another aspect of present disclosure, the crystalline salts also relates to co-crystals of Daprodustat.

In another aspect, the present disclosure provides a novel synthetic process for preparing Daprodustat involving intermediate of formula 2 and formula 3.

In another aspect of the present disclosure, the synthetic process is summarized in Scheme 1.

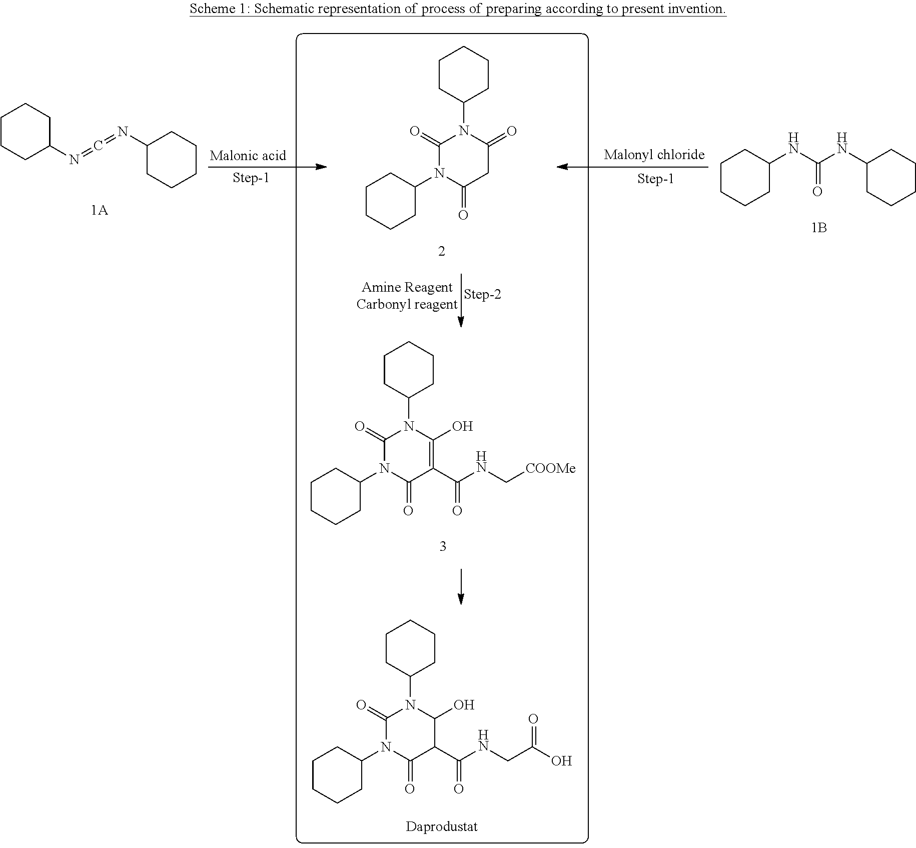 [SMILES: C(=NC1CCCCC1)=NC1CCCCC1 ; O=C1CC(=O)N(C2CCCCC2)C(=O)N1C1CCCCC1 ; O=C(NC1CCCCC1)NC1CCCCC1 ; COC(=O)CNC(=O)c1c(O)n(C2CCCCC2)c(=O)n(C2CCCCC2)c1=O ; O=C(O)CNC(=O)C1C(=O)N(C2CCCCC2)C(=O)N(C2CCCCC2)C1O]

In another aspect, the present disclosure provides a novel process for preparing Daprodustat, the process including:
a) Converting a compound 2 to compound 3 in presence of in presence of carbonyl reagent (C═O reagent) and amine reagent
b) Converting the compound 3 to Daprodustat.

Step a) can be achieved by reaction of compound 2 with carbonyl reagent followed by amine reagent or reaction of carbonyl reagent with the amine reagent followed by further reaction with compound 2. Compound of formula 3 can be optionally be isolated as solid and PXRD is given in FIG. 25.

In another aspect, the present disclosure involves the process for preparation of Daprodustat, the process including:
a) Converting compound 2 to compound 3 in presence of carbonyldiimidazole and Glycine methylester,
b) Converting the compound 3 to Daprodustat.

Step a) can be achieved by first reaction of compound 2 with carbonyldiimidazole, followed by Glycine methylester or first reaction of carbonyldiimidazole with Glycine methylester, followed by reaction with compound 2.

In another aspect of present disclosure, the reaction further involves preparing Daprodustat from compound of formula 3.

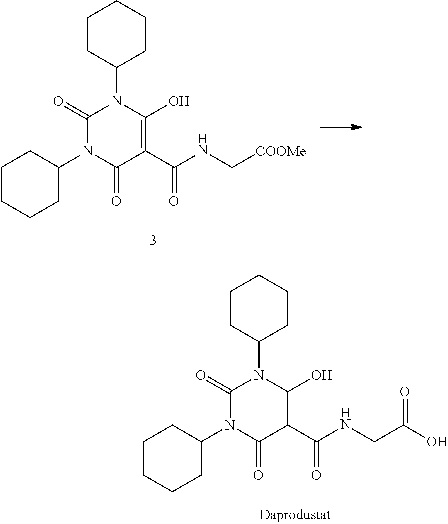 [SMILES: COC(=O)CNC(=O)c1c(O)n(C2CCCCC2)c(=O)n(C2CCCCC2)c1=O ; O=C(O)CNC(=O)C1C(=O)N(C2CCCCC2)C(=O)N(C2CCCCC2)C1O]

In another aspect, the present disclosure provides novel intermediates of formula 2A and Formula 2B which are formed in Step a) of the above process.

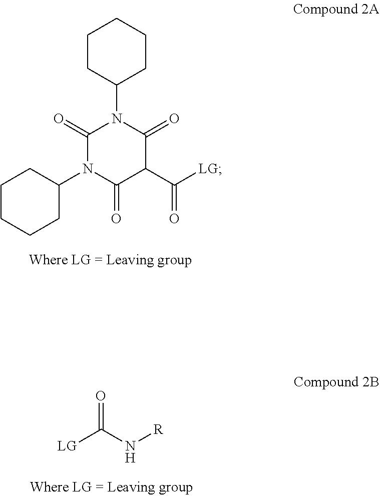

where R includes but not limited to —CH₂COOR1, CH₂COOH, —CH₂CN, —CH₂CONH₂, where R1 is —CH₃ to C₇H₁₅

In another aspect, the present disclosure provides novel intermediate of following formula.

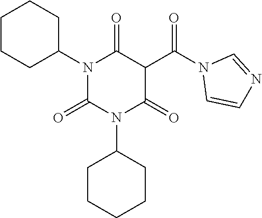 [SMILES: O=C1C(C(=O)n2ccnc2)C(=O)N(C2CCCCC2)C(=O)N1C1CCCCC1]

In another aspect, the present disclosure provides novel intermediate of formula 3.

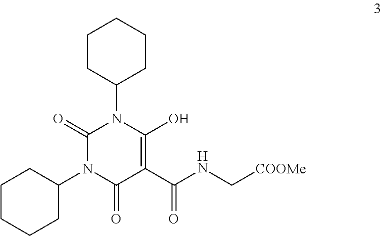 [SMILES: COC(=O)CNC(=O)c1c(O)n(C2CCCCC2)c(=O)n(C2CCCCC2)c1=O]

Compound of formula 3 is optionally isolated as solid, either as amorphous or crystalline form, preferably as crystalline form. PXRD of isolated crystalline form is given in FIG. 25.

In another aspect, the present disclosure provides a crystalline Form of Daprodustat, and its the pharmaceutical compositions thereof, comprising Daprodustat with a chemical purity of at least 99% by HPLC or at least 99.5% by HPLC or at least 99.9% by HPLC.

In another aspect, the present disclosure provides the control of impurities of following formula below 0.15% wt/wt by HPLC method.

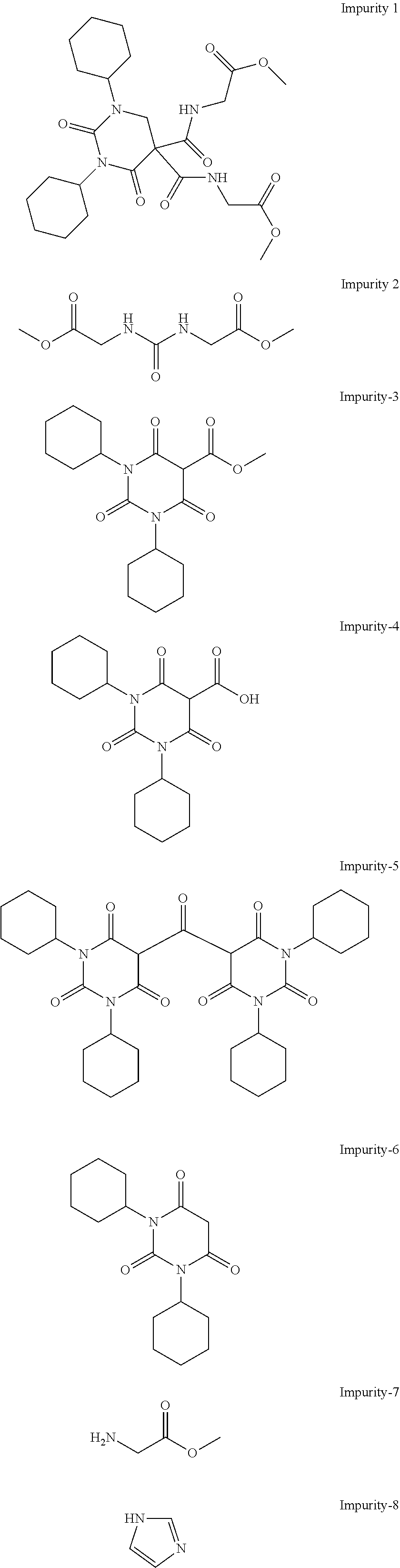

In certain embodiment(s) of the present disclosure, the term “amorphous” refers to solid forms that consist of disordered arrangements of molecules and do not possess a distinguishable crystal lattice.

### Definitions

In certain embodiment(s) of the present disclosure, the term “crystalline” refers to compounds or compositions where the structural units are arranged in fixed geometric patterns or lattices, so that crystalline solids have rigid long range order. The structural units that constitute the crystal structure can be atoms, molecules, or ions. Crystalline solids show definite melting points.

In certain embodiment(s) of the present disclosure, the term “dispersion” refers to a disperse system in which one substance, the dispersed phase, is distributed, in discrete units, throughout a second substance (the continuous phase or vehicle). The size of the dispersed phase can vary considerably (e.g. colloidal particles of nanometer dimension, to multiple microns in size). In general, the dispersed phases can be solids, liquids, or gases. In the case of a solid dispersion, the dispersed and continuous phases are both solids. In pharmaceutical implementations, a solid dispersion can include a crystalline drug (dispersed phase) in an amorphous polymer (continuous phase), or alternatively, an amorphous drug (dispersed phase) in an amorphous polymer (continuous phase).

In certain embodiment(s) of the present disclosure, the term “amorphous solid dispersion” generally refers to a solid dispersion of two or more components, usually a drug and polymer, optionally containing other components such as surfactants or other pharmaceutical excipients, where drug is either amorphous or crystalline, and the physical stability and/or dissolution and/or solubility of the amorphous drug is enhanced by the other components.

In certain embodiment(s) of the present disclosure, the term “about” when used in the present disclosure preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

In certain embodiment(s) of the present disclosure, the term “carbonyl reagent (C═O reagent)” used in the present disclosure refers to any reagent which imparts carbonyl group to the target site. Such reagents include but not limited to carbonyldiimidazole, phosgene, diphosgene, triphosgene, N,N′-disuccinimidyl carbonate, bis(pentafluorophenyl)carbonate, bis(4-nitrophenyl)carbonate, 4-nitrophenylcarbonate and like.

In certain embodiment(s) of the present disclosure, the term ‘Amine reagent (R—NH2 reagent)” used in the present disclosure refers to any reagent that will impart the necessary functional group, where R includes but not limited to —CH2COOR1, CH2COOH, —CH2CN, —CH2CONH2, where R1 is —CH3 to C7H15. The amine reagents include but not limited to glycine methylester, glycine ethylester, glycine isopropylester, glycine benzylester, glycine tertbutylester, glycine n-propoyl ester and like, or their inorganic and organic salts thereof.

In certain embodiment(s) of the present disclosure, the term “base” used in the present disclosure refers to inorganic and organic base.

In certain embodiment(s) of the present disclosure, the term “organic base” used in the present disclosure includes but not limited to triethylamine, pyridine, DBU, DABCO, DIPEA, DMAP, NaOMe, NaH, NaOEt, tBuOK, BuLi, tBuLi, LHMDS, imidazole and like; The inorganic base used in the present disclosure includes but not limited to Cs2CO3, K2CO3, NaHCO₃, NaOH, KOH, LiOH, Na2CO3 and like or mixture thereof.

In certain embodiment(s) of the present disclosure, suitable “organic solvent” include, but are not limited to, alcohol, aliphatic hydrocarbon, alicyclic hydrocarbon, aromatic hydrocarbon, halogenated hydrocarbon, ester, ether, nitrile, polar aprotic, ketone, water or mixtures thereof.

In certain embodiment(s) of the present disclosure, an “alcohol solvent” is an organic solvent containing a carbon bound to a hydroxyl group. “Alcoholic solvents” include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, Ci-6 alcohols, or mixtures thereof.

In certain embodiment(s) of the present disclosure, an “aliphatic or alicyclic hydrocarbon solvent” refers to a liquid, non-aromatic, hydrocarbon, which may be linear, branched, or cyclic. It is capable of dissolving a solute to form a uniformly dispersed solution. Examples of a hydrocarbon solvents include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, C5-C8 aliphatic hydrocarbons, petroleum ethers, or mixtures thereof.

In certain embodiment(s) of the present disclosure, “aromatic hydrocarbon solvent” refers to a liquid, unsaturated, cyclic, hydrocarbon containing one or more rings which has at least one 6-carbon ring containing three double bonds. It is capable of dissolving a solute to form a uniformly dispersed solution. Examples of aromatic hydrocarbon solvents include, but are not limited to, benzene toluene, ethylbenzene, m-xylene, o-xylene, p-xylene, indane, naphthalene, tetralin, trimethylbenzene, chlorobenzene, fluorobenzene, trifluorotoluene, anisole, C6-C10aromatic hydrocarbons, or mixtures thereof.

In certain embodiment(s) of the present disclosure, an “ester solvent” is an organic solvent containing a carboxyl group —(C═O)—O— bonded to two other carbon atoms. “Ester solvents” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, C3-6 esters, or mixtures thereof.

In certain embodiment(s) of the present disclosure, a “halogenated hydrocarbon solvent” is an organic solvent containing a carbon bound to a halogen. “Halogenated hydrocarbon solvents” include, but are not limited to, dichloromethane, 1,2-dichloroethane, trichloroethylene, perchloroethylene, 1,1,1-trichloroethane, 1,1,2-trichloroethane, chloroform, carbon tetrachloride, or mixtures thereof.

In certain embodiment(s) of the present disclosure, a “ketone solvent” is an organic solvent containing a carbonyl group —(C═O)— bonded to two other carbon atoms. “Ketone solvents” include, but are not limited to, acetone, ethyl methyl ketone, diethyl ketone, methyl isobutyl ketone, C3-6 ketones, 4-methyl-pentane-2-one or mixtures thereof.

In certain embodiment(s) of the present disclosure, a “nitrile solvent” is an organic solvent containing a cyano-(CEN) bonded to another carbon atom. “Nitrile solvents” include, but are not limited to, acetonitrile, propionitrile, C2-6 nitriles, or mixtures thereof.

In certain embodiment(s) of the present disclosure, a “polar aprotic solvent” has a dielectric constant greater than 15 and is at least one selected from the group consisting of amide-based organic solvents, such as N,N-dimethylformamide (DMF), N, N-dimethylacetamide (DMA), N-methylpyrrolidone (NMP), formamide, acetamide, propanamide, hexamethyl phosphoramide (HMPA), and hexamethyl phosphorus triamide (HMPT); nitro-based organic solvents, such as nitromethane, nitroethane, nitropropane, and nitrobenzene; pyridine-based organic solvents, such as pyridine and picoline; sulfone-based solvents, such as dimethylsulfone, diethylsulfone, diisopropylsulfone, 2-methylsulfolane, 3-methylsulfolane, 2,4-dimethylsulfolane, 3,4-dimethy sulfolane, 3-sulfolene, and sulfolane; and sulfoxide-based solvents such as dimethylsulfoxide (DMSO).

In certain embodiment(s) of the present disclosure, an “ether solvent” is an organic solvent containing an oxygen atom —O— bonded to two other carbon atoms. “Ether solvents” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, C2-6 ethers, or the like. or aqueous media including water or aqueous buffers or mixture of them.

### Examples

### Example-1: Preparation of Daprodustat ASD with Copovidone (1:3)

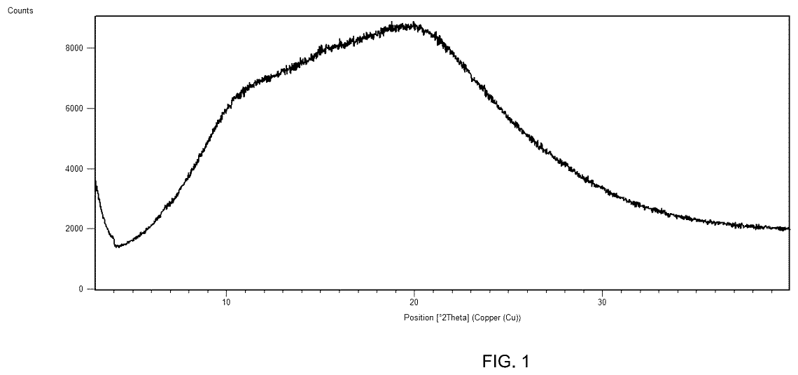

The Daprodustat (1 g) and copovidone (3 g) are dissolved in acetone (80 ml) at 35-40° C. Filtered the solution to get the particle free and evaporated the solution over spray drier at 65-70° C. The resultant solid PXRD is given in FIG. 1.

### Example-2: Preparation of Daprodustat ASD with HPMC (1:3)

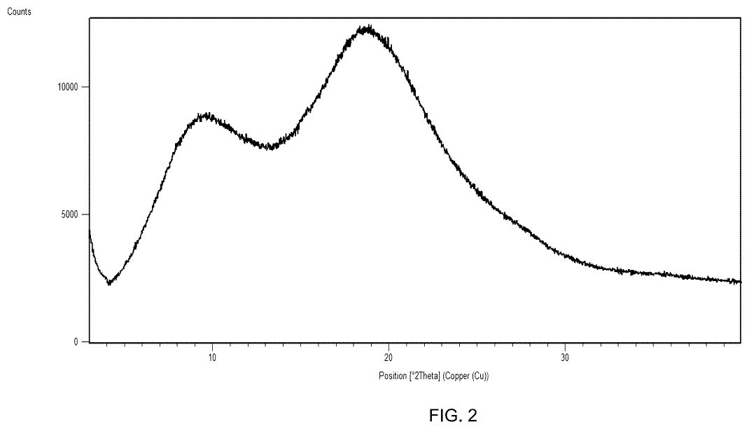

Daprodustat (1 g) and HPMC (3 g) are dissolved in mixture of methanol (200 mL) and dichloromethane (100 mL) at 25-30° C. Filtered the solution to get the particle free and evaporated the solution over spray drier at 65-70° C. The PXRD of the resultant solid material is given in FIG. 2.

### Example-3: Preparation of Crystalline Form-DP1 of Daprodustat

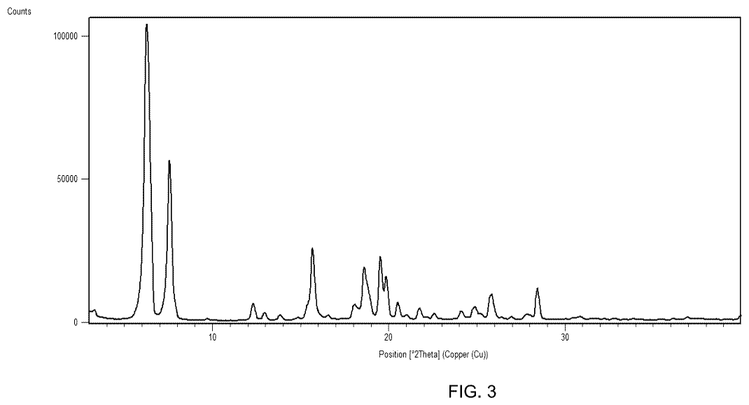

The Daprodustat (1 g) is suspended in MTBE (20 mL) and stirred reaction mass at 25-30° C. about 20-22 hours. The solid material obtained is filtered under vacuum to obtain crystalline Form-DP1 of Daprodustat. The PXRD of Form DP-1 is given in FIG. 3.

PXRD (2θ) characteristic peaks for Form DP1: 6.29, 7.56, 12.30, 12.94, 13.81, 15.68, 16.53, 18.07, 18.59, 19.52, 19.84, 20.50, 21.73, 24.09, 24.84, 25.79

### Example-4: Preparation of Crystalline Form-DP2 of Daprodustat

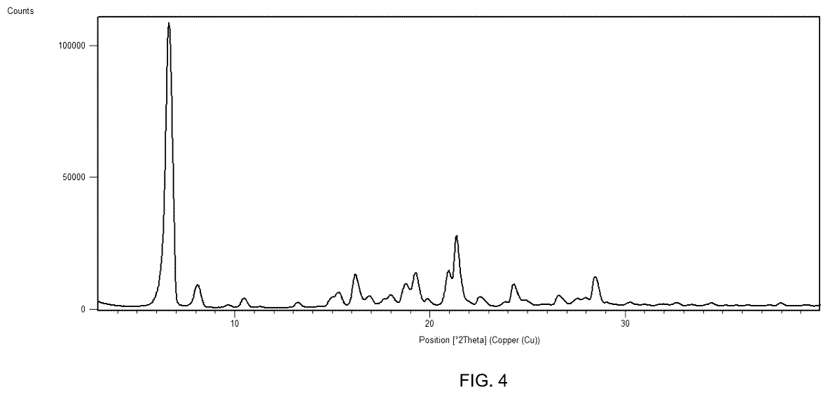

The Daprodustat (1 g) is suspended in THF (15 mL) and stirred reaction mass at 25-30° C. about 1-2 hours. The solid material obtained is filtered under vacuum to obtain crystalline Form-DP2 of Daprodustat. The PXRD of Form DP-2 is given in FIG. 4.

PXRD (2θ) characteristic peaks for Form DP2: 6.64, 8.09, 9.64, 13.22, 15.31, 16.19, 16.86, 17.98, 18.73, 19.25, 20.91, 21.37, 22.62, 24.29, 26.61

### Example-5: Preparation of Crystalline Form-DP3 of Daprodustat

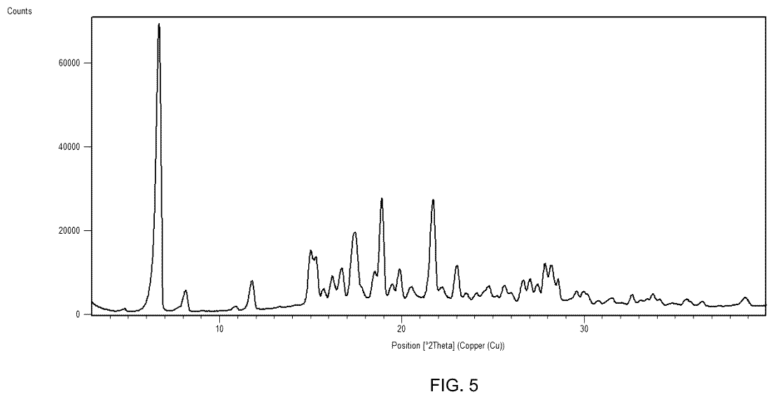

The Daprodustat (0.25 g) is suspended in NMP (2.5 mL) and stirred reaction mass at 25-30° C. for about 1-2 days. The solid material obtained is filtered under vacuum to obtain crystalline Form-DP3 of Daprodustat. The PXRD of Form DP-3 is given in FIG. 5.

PXRD (2θ) characteristic peaks for Form DP3: 4.60, 6.70, 8.14, 10.85, 11.77, 15.0, 16.19 16.70, 17.44, 18.89, 19.87, 20.49, 21.70, 23.00, 25.60, 27.85.

### Example-6: Preparation of Crystalline Form-DP4 of Daprodustat

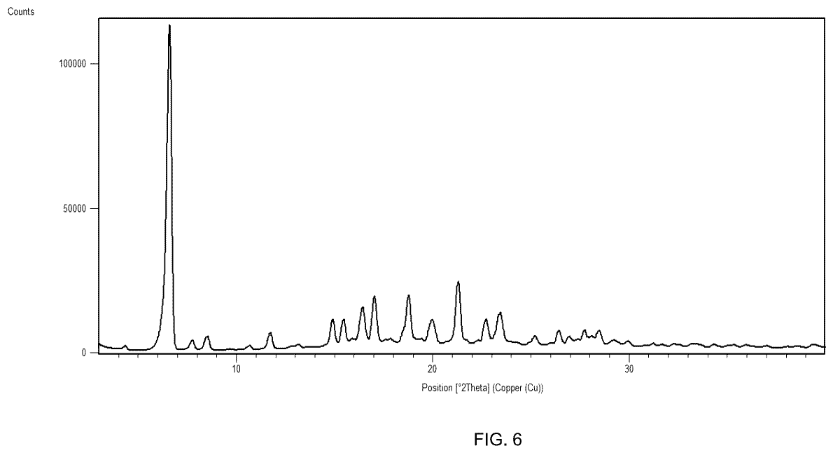

The Daprodustat (0.25 g) is suspended in DMAC (2.5 mL) and stirred reaction mass at 25-30° C. for about 1-2 days. The solid material obtained is filtered under vacuum to obtain crystalline Form-DP4 of Daprodustat. The PXRD of Form DP-4 is given in FIG. 6.

PXRD (2θ) characteristic peaks for Form DP4: 4.30, 6.59, 7.72, 8.50, 10.61, 11.71, 13.10, 14.90, 15.44, 16.41, 17.02, 18.76, 19.95, 21.28, 22.69, 23.40, 25.16, 26.40, 27.70.

### Example-7: Preparation of Crystalline Form-DP5 of Daprodustat

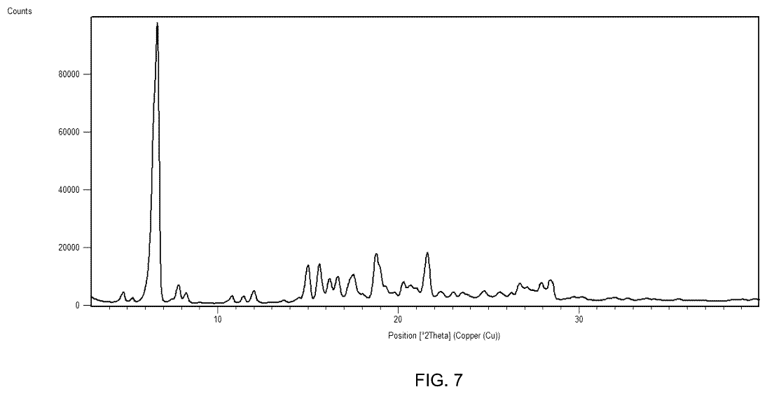

The Daprodustat (0.25 g) is suspended in DMF (2.5 mL) and stirred reaction mass at 25-30° C. for about 1-2 days. The solid material obtained is filtered under vacuum to obtain crystalline Form-DP5 of Daprodustat. The PXRD of Form DP-5 is given in FIG. 7.

PXRD (2θ) characteristic peaks for Form DP5: 4.74, 5.25, 6.67, 7.81, 8.24, 10.74, 11.41, 11.98, 14.97, 15.63, 16.18, 16.63, 17.44, 18.74, 20.26, 21.57, 23.00, 24.71, 25.59, 26.69, 27.91.

### Example-8: Preparation of Crystalline Form-DP6 of Daprodustat

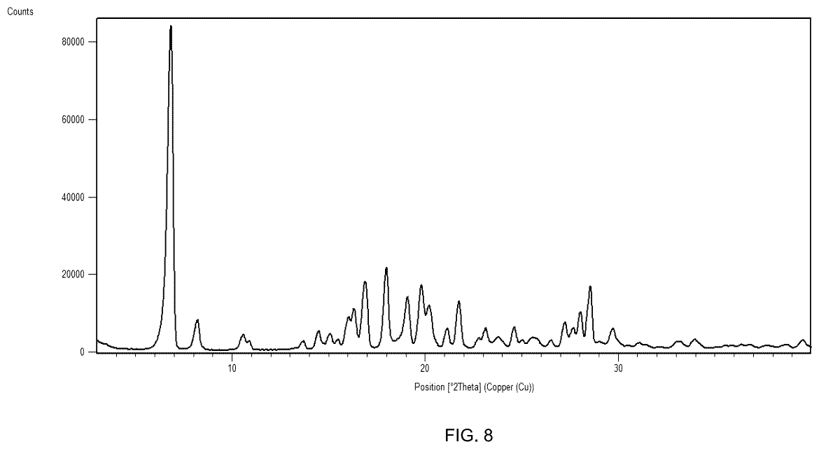

The Daprodustat (0.25 g) is suspended in 1,4 dioxane (2.5 mL) and stirred reaction mass at 25-30° C. for about 1-2 days. The solid material obtained is filtered under vacuum to obtain crystalline Form-DP6 of Daprodustat. The PXRD of Form DP-6 is given in FIG. 8.

PXRD (2θ) characteristic peaks for Form DP6: 6.83, 8.18, 10.88, 13.64, 14.46, 15.04, 16.30, 16.90, 17.97, 19.07, 19.79, 21.11, 21.72, 23.09, 24.58, 26.46, 27.20, 28.01, 29.69.

### Example-9: Preparation of Crystalline Form-DP7 of Daprodustat

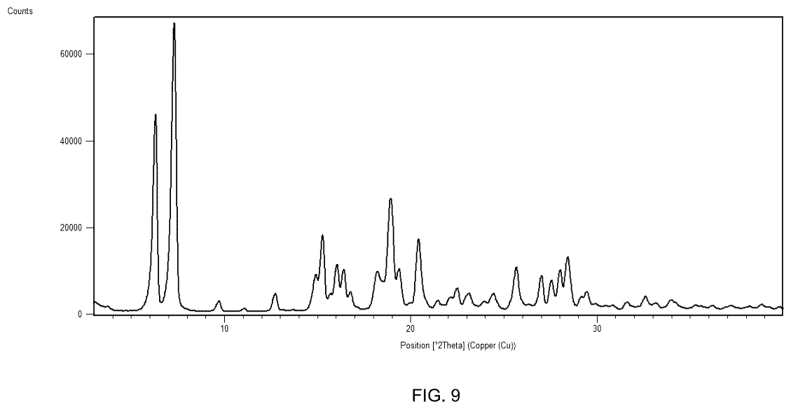

The Daprodustat (0.25 g) is suspended in 2-methoxy ethanol (2.5 mL) and stirred reaction mass at 25-30° C. for about 1-2 days. The solid material obtained is filtered under vacuum to obtain crystalline Form-DP7 of Daprodustat. The PXRD of Form DP-7 is given in FIG. 9.

PXRD (2θ) characteristic peaks for Form DP7: 6.28, 7.29, 9.68, 11.03, 12.71, 15.25, 16.01, 16.39, 18.22, 18.90, 20.41, 21.43, 22.47, 23.08, 24.40, 25.64, 27.00, 27.53, 28.00, 29.43.

### Example-10: Preparation of Crystalline Form-DP8 of Daprodustat

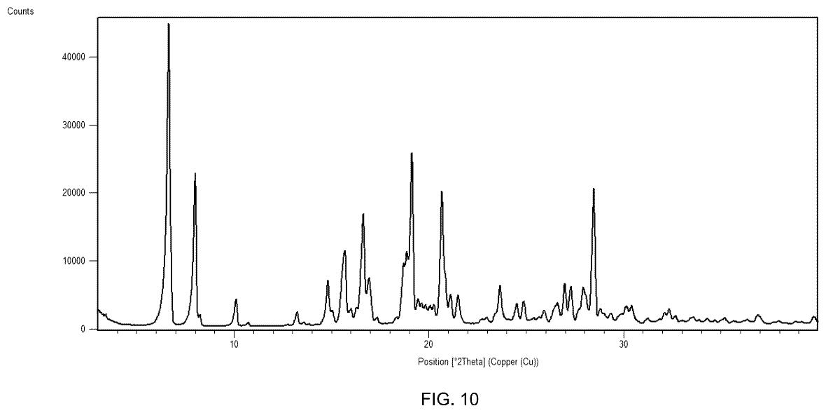

The Daprodustat (0.5 g) is suspended in DMSO (6 mL) and stirred reaction mass at 25-30° C. for about 1-2 days. The solid material obtained is filtered under vacuum to obtain crystalline Form-DP8 of Daprodustat. The PXRD of Form DP-8 is given in FIG. 10.

PXRD (2θ) characteristic peaks for Form DP8: 6.63, 8.00, 10.10, 13.22, 14.80, 15.68, 16.00, 16.63, 16.92, 18.86, 19.13, 20.65, 21.10, 21.49, 23.64, 24.51, 24.85, 26.90, 27.28, 27.90, 30.39.

### Example-11: Preparation of Crystalline Form-DP9 of Daprodustat

Form DP8 obtained from Example 10 is dried in a vacuum tray dryer at 55-60° C. about 40-45 hrs to obtain Form DP-9 of Daprodustat and the PXRD of Form DP-9 is given in FIG. 11.

PXRD (2θ) characteristic peaks for Form DP9: 6.38, 7.18, 7.52, 10.78, 12.25, 12.92, 15.36, 16.65, 17.18, 18.06, 18.67, 19.14, 19.72, 20.84, 22.73, 24.78, 26.94, 27.89.

### Example-12: Preparation of Daprodustat ASD with Eudragit (1:9)

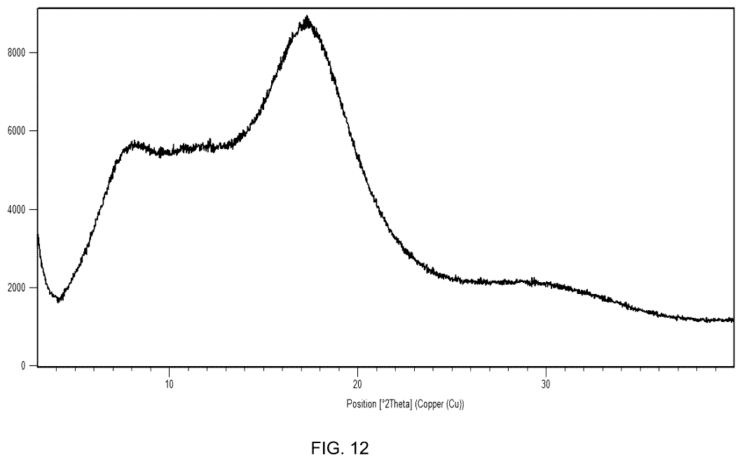

The Daprodustat (1 g) and Eudragit (9 g) are dissolved in methanol (50 ml) and 50 mL DCM at 35-40° C. Filtered the solution to get the particle free and evaporated the solution over spray drier at 65-70° C. The resultant solid PXRD is given in FIG. 12.

### Example-13: Preparation of Daprodustat ASD with HPC (1:9)

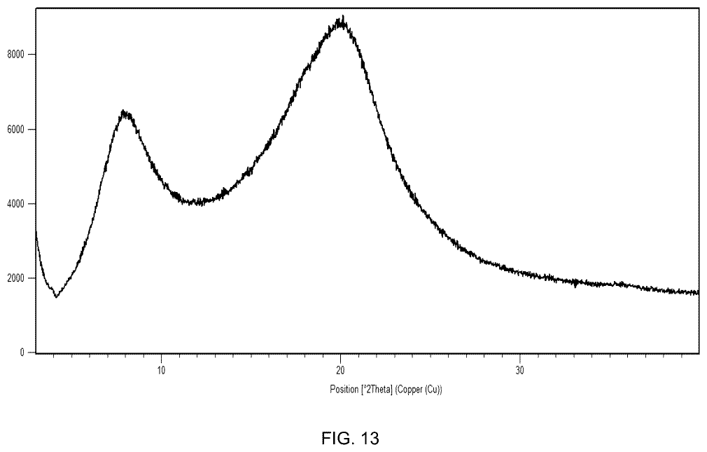

Daprodustat (1 g) and Hydroxypropylcellulose (HPC) (9 g) are dissolved in mixture of 75 mL of methanol and 75 mL DCM at 35-40° C. Filtered the solution to get the particle free and evaporated the solution over spray drier at 65-70° C. The PXRD of the resultant solid material is given in FIG. 13.

### Example-14: Preparation of Daprodustat ASD with HPMC (1:9)

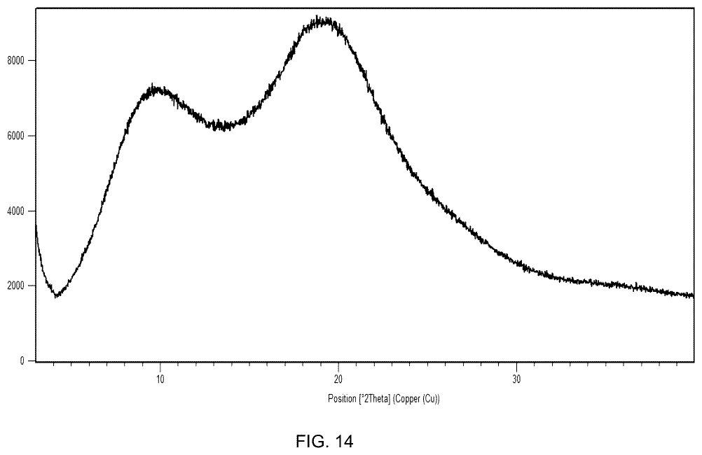

Daprodustat (1 g) and Hydroxypropyl methylcellulose (HPMC) (9 g) are dissolved in mixture of 75 mL of methanol and 75 mL DCM at 35-40° C. Filtered the solution to get the particle free and evaporated the solution over spray drier at 65-70° C. The PXRD of the resultant solid material is given in FIG. 14.

### Example-15: Preparation of Daprodustat ASD with HPMC-AS (1:9)

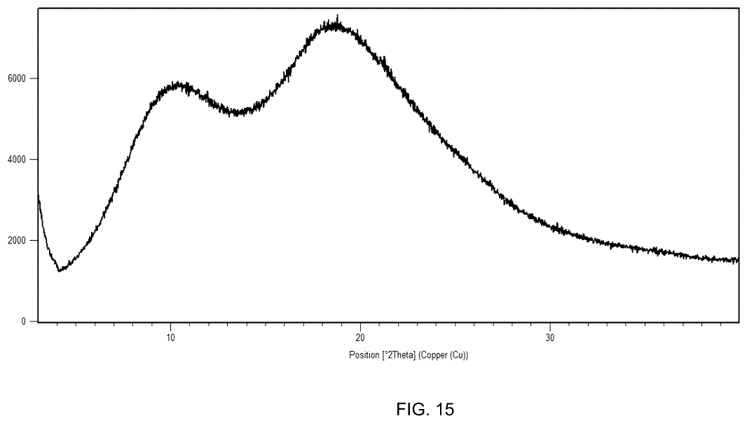

Daprodustat (1 g) and hydroxypropyl methylcellulose acetate succinate (HPMC AS) (9 g) are dissolved in mixture of 75 mL of methanol and 75 mL DCM at 35-40° C. Filtered the solution to get the particle free and evaporated the solution over spray drier at 65-70° C. The PXRD of the resultant solid material is given in FIG. 15.

### Example-16: Preparation of Daprodustat ASD with PVPK-90 (1:9)

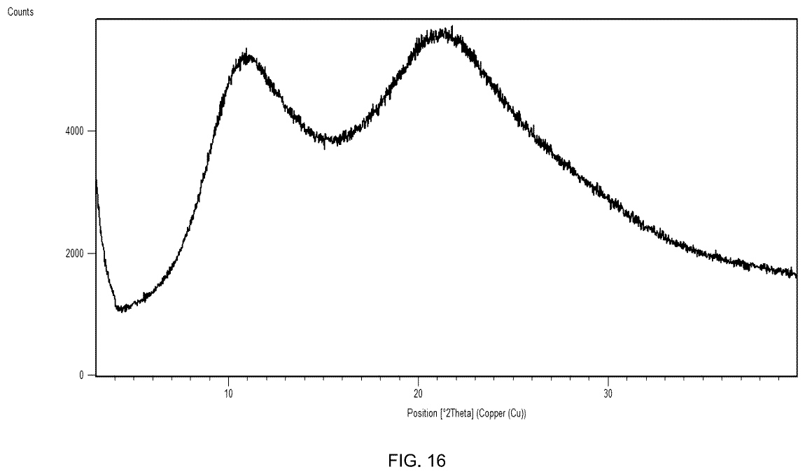

Daprodustat (1 g) and Polyvinylpyrrolidone (PVP) K-90 (9 g) are dissolved in mixture of 75 mL of methanol and 75 mL DCM at 35-40° C. Filtered the solution to get the particle free and evaporated the solution over spray drier at 65-70° C. The PXRD of the resultant solid material is given in FIG. 16.

### Example-17: Preparation of Daprodustat ASD with Copovidone (1:9)

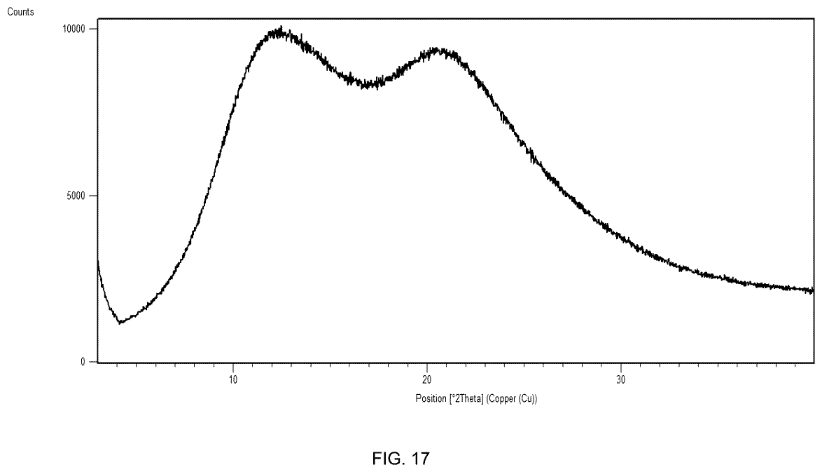

Daprodustat (1 g) and Copovidone (9 g) are dissolved in mixture of 50 mL of methanol and 50 mL DCM at 35-40° C. Filtered the solution to get the particle free and evaporated the solution over spray drier at 65-70° C. The PXRD of the resultant solid material is given in FIG. 17.

### Example-18: Preparation of Crystalline Form-DP10 of Daprodustat

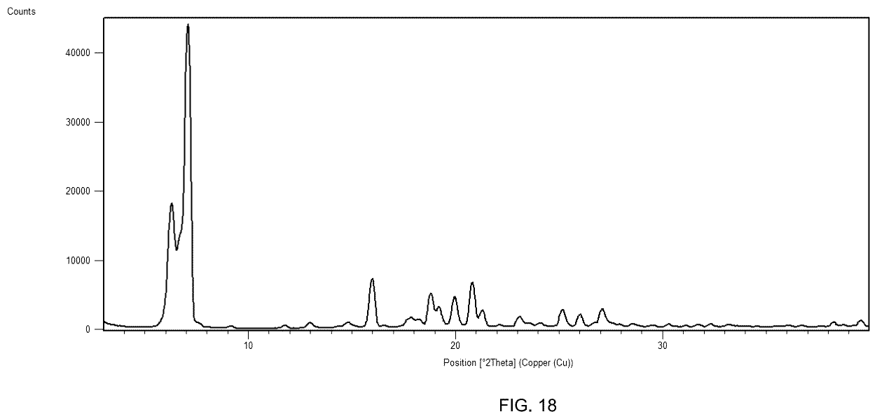

The Daprodustat (0.25 g) is suspended in 2-butanol (2.5 mL) and stirred reaction mass at −5 to 0° C. about 1-2 days. The solid material obtained is filtered under vacuum to obtain crystalline Form-DP10 of Daprodustat. The PXRD of Form DP-10 is given in FIG. 18.

PXRD (2θ) characteristic peaks for Form DP10: 6.3, 6.72, 7.08, 9.15, 11.73, 12.95, 14.79, 15.97, 17.85, 18.79, 19.18, 19.96, 20.79, 21.27, 23.05, 24.06, 25.15, 25.99, 27.07

### Example-19: Preparation of Crystalline Form-DP11 of Daprodustat

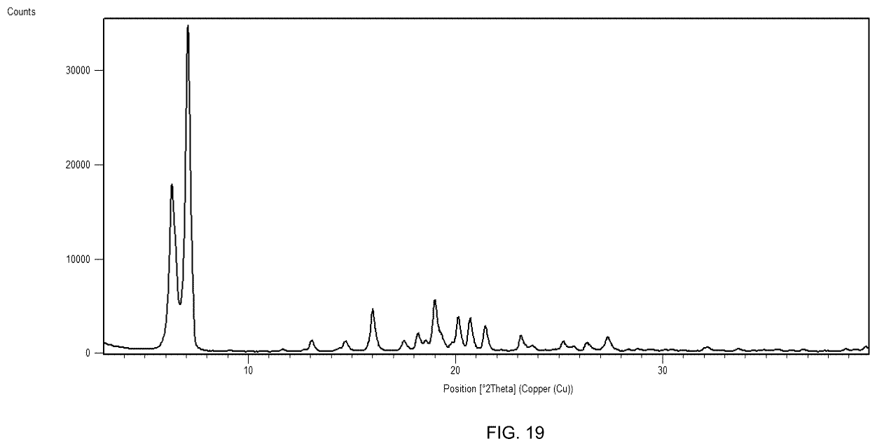

The Daprodustat (0.25 g) is suspended in ter-butyl alcohol (2.5 mL) and stirred reaction mass at −5 to 0° C. about 1-2 days. The solid material obtained is filtered under vacuum to obtain crystalline Form-DP11 of Daprodustat. The PXRD of Form DP-11 is given in FIG. 19.

PXRD (2θ) characteristic peaks for Form DP11: 6.35, 7.08, 9.02, 11.63, 13.05, 14.66, 16.01, 17.50, 18.19, 18.53, 19.04, 19.81, 20.14, 20.71, 21.44, 22.19, 23.16, 23.64, 25.19, 25.65, 26.37, 27.34.

### Example-20: Preparation of Crystalline Form of Daprodustat with Nicotinamide

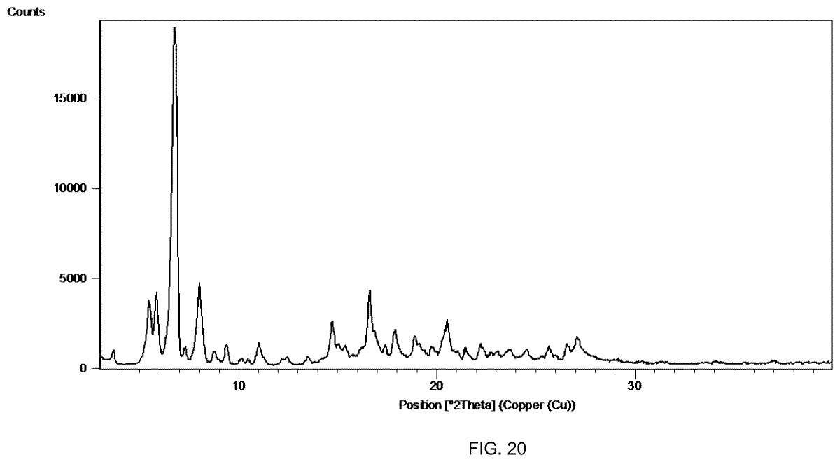

The Daprodustat (1 g) and Nicotinamide (0.9 g) are suspended in acetone (15 ml) and stirred reaction mass at 25-30° C. for about 1-3 days. The material is filtered under vacuum to obtained title compound. The resultant solid PXRD is given in FIG. 20 (Form CC1).

PXRD (2θ): 3.67, 5.48, 5.86, 6.75, 7.30, 8.02, 8.79, 9.38, 10.14, 10.48, 11.03, 12.47, 13.48, 14.73, 15.37, 16.62, 17.38, 17.92, 18.99, 20.52, 21.48, 22.26, 23.64, 24.51, 25.67, 26.59, 27.14 2θ.

### Example-21: Preparation of Crystalline Form of Daprodustat with Caffeine

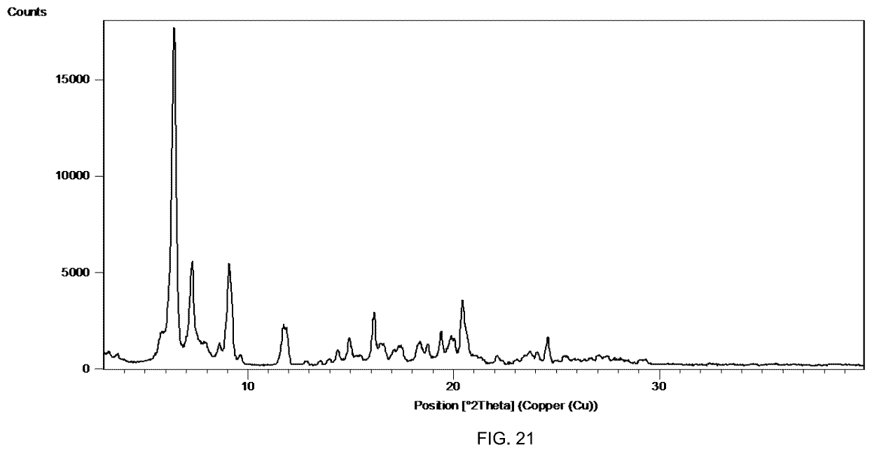

The Daprodustat (1 g) is suspended in Isopropyl alcohol (15 ml) and Caffeine (0.25 g) dissolved in acetone (0.5 ml) is slowly added. Stirred reaction mass at 25-30° C. for about 1-3 days. The material is filtered under vacuum to obtained title compound. The resultant solid PXRD is given in FIG. 21 (Form CC2).

PXRD (2θ): 3.25, 6.41, 7.29, 9.11, 9.65, 11.73, 12.83, 13.52, 13.96, 14.38, 14.95, 15.36, 16.14, 16.47, 17.41, 18.43, 18.75, 19.41, 19.87, 20.5, 22.14, 24.08, 24.58 2θ.

### Example-22: Preparation of Crystalline Form of Daprodustat with L-Proline

The Daprodustat (1 g) and L-Proline (0.3 g) is suspended in acetone (25 ml). Stirred reaction mass at 25-30° C. for about 5-8 hours. The material is filtered under vacuum to obtained title compound. The resultant solid PXRD peaks are given below.

PXRD (2θ): 3.19, 6.57, 7.19, 8.06, 9.16, 9.82, 10.42, 11.75, 13.15, 13.76, 14.63, 15.28, 16.17, 17.29, 17.71, 18.43, 19.43, 19.77, 20.46, 20.94, 22.29, 23.12, 23.69, 24.02, 25.07, 26.55 2θ.

### Example-23: Preparation of Crystalline Form of Daprodustat with L-Proline

Form CC3A obtained in Example 3A is dried in vacuum tray dryer and The resultant solid PXRD peaks are given below (Form CC3B).

PXRD (2θ): 3.48, 5.18, 6.59, 6.92, 7.26, 8.23, 8.63, 9.36, 10.2, 10.67, 12.12, 12.79, 13.74, 15.26, 15.76, 16.39, 16.95, 17.25, 18.11, 18.99, 19.28, 20.07, 20.49, 21.15, 21.71, 23.67, 24.66, 27.09, 27.97 2θ.

### Example-24: Preparation of Crystalline Form of Daprodustat with Aspartame

The Daprodustat (0.1 g) and Aspartame (0.3 g) are suspended in acetone (9 ml) and stirred reaction mass at 25-30° C. for about 1-2 days. The material is filtered under vacuum to obtained title compound. The resultant solid PXRD peaks are given below (Form CC4).

PXRD (2θ): 4.92, 6.99, 9.85, 11.09, 12.24, 13.15, 14.07, 14.96, 15.75, 17.04, 17.98, 18.66, 19.4, 19.82, 20.60, 21.20, 22.38, 22.9, 23.49, 24.02, 25.06, 25.57, 26.98, 27.52, 27.95, 28.9, 30.63, 32.72 2θ.

### Example-25: Preparation of Crystalline Form of Daprodustat with Urea

Daprodustat (0.25 g) and Urea (0.25) are suspended are suspended in Methanol (10 ml) and stirred reaction mass at 25-30° C. for about 3-5 days. The material is filtered under vacuum to obtain title compound. The resultant solid PXRD peaks are given below (Form CC5).

PXRD (2θ): 3.79, 6.52, 7.58, 11.25, 12.89, 15.44, 16.25, 16.80, 18.82, 19.73, 20.10, 21.03, 22.33, 23.02, 23.54, 24.88, 26.30, 27.05, 28.11, 29.37, 30.16, 33.70 2θ.

### Example-26: Preparation of 1,3-dicyclohexylpyrimidine-2,4,6 (1H,3H,5H)-trione (2)

### Method-1

A solution of N,N-dicyclohexylcarbodiimide (40 g; 194 mmol) in anhydrous THF (80 mL) is added dropwise to a solution of malonic acid (10 g; 97 mmol.) in anhydrous THF (60 mL) at 0-5° C. The mixture is stirred and allowed to warm to ambient temperature over 2 h. The reaction mass is filtered and washed with THF (20 mL). The filtrate is distilled under vacuum to obtain a yellow solid, which is slurried in ethanol (150 mL) and heated to reflux temperature. The mixture is then allowed to cool to ambient temperature and filtered. The filtered solid is washed with cold ethanol (20 mL) and dried under vacuum to afford the title compound as a off white colour solid with purity >99.0%. 1H NMR (400 MHz, DMSO-d6): δ ppm 4.47-4.42 (m, 2H), 3.68 (s, 2H), 2.12 (q, J=12.5, 10.0 Hz, 4H), 1.75 (d, J=12.5 Hz, 4H), 1.61-1.55 (m, 6H), 1.24 (q, J=13.0 Hz, 4H), 1.13-1.06 (m, 2H). Mass: m/z: 291.1 [M−H]+, Theoretical exact mass of 2:292.17 (Mol Formula: C16H24N2O3)

### Method-2

A solution of N,N-dicyclohexylcarbodiimide (40 g; 194 mmol) in DCM (100 mL) is added dropwise to a suspension of malonic acid (10 g; 97 mmol) in DCM (2θ0 mL) at 25-35° C. The mixture is stirred for over 2 h. The reaction mass is filtered and washed with DCM (50 mL). The filtrate is distilled under vacuum to obtain a yellow solid, which is slurried in methanol (180 mL) and heated to reflux temperature. The mixture is then allowed to cool to 0-10° C., maintained for 2-4 hrs and filtered. The filtered solid is washed with cold methanol (2*50 mL) and dried under vacuum to afford the title compound as a off white colour solid with purity >99.0%. 1H NMR (400 MHZ, DMSO-d6): δ ppm 4.45-4.41 (m, 2H), 3.68 (s, 2H), 2.17-2.08 (m, 4H), 1.76 (d, J=13 Hz, 4H), 1.58 (t, J=15 Hz, 13 Hz, 6H), 1.28-1.21 (m, 4H), 1.14-1.12 (m, 2H). Mass: m/z: 293.0 [M+H]+, Theoretical exact mass of 2:292.17 (Mol Formula: C16H24N2O3)

### Method-3

A solution of N,N-dicyclohexylcarbodiimide (40 g; 194 mmol) in DMF (120 mL) is added dropwise to a solution of malonic acid (10 g; 97 mmol) in DMF (50 mL) at 25-35° C. The mixture is stirred over 2 h. The reaction mass is filtered and washed with DMF (50 mL). The filtrate is distilled under vacuum to obtain a yellow solid, which is slurried in methanol (100 mL) and heated to reflux temperature. The mixture is then allowed to cool to 0-10° C., maintained for 2-4 hrs and filtered. The filtered solid is washed with cold methanol (2*50 mL) and dried under vacuum to afford the title compound as a off white to yellow colour solid.

### Method-4

A solution of N, N-dicyclohexylcarbodiimide (40 g; 194 mmol) in Toluene (120 mL) is added dropwise to a solution of malonic acid (10 g; 97 mmol) in Toluene (50 mL) at 25-35° C. The mixture is stirred over 2 h. The reaction mass is filtered and washed with Toluene (50 mL). The filtrate is distilled under vacuum to obtain a yellow solid, which is slurried in methanol (100 mL) and heated to reflux temperature. The mixture is then allowed to cool to 0-10° C., maintained for 2-4 hrs and filtered. The filtered solid is washed with cold methanol (2*50 mL) and dried under vacuum to afford the title compound as a off white colour solid.

### Example-27: Preparation of methyl (1,3-dicyclohexyl-6-hydroxy-2,4-dioxo-1,2,3,4-tetrahydropyrimidine-5-carbonyl)glycinate

### Method-1

In a round bottomed flask, glycine methyl ester hydrochloride (0.94 g, 7.48 mmol) in THF (10 mL) is added to a solution of 1,3-dicyclohexylpyrimidine-2,4,6 (1H,3H,5H)-trione (10 g, 6.8 mmol), N,N-Diisopropylethylamine (1.76 g, 13.6 mmol) and 1,1′-Carbonyldiimidazole (1.1 g, 6.8 mmol) in anhydrous THF (20 mL) at 25-35° C. The solution is stirred at 25-35° C. for 1-2 hr. After completion of reaction, water (20 mL) is added to the reaction mass to obtain a solid precipitate. The obtained solid is filtered and dried under vacuum to obtain N-[(1,3-dicyclohexylhexahydro-2,4,6-trioxo-5-pyrimidinyl)carbonyl]glycine methyl ester as off-white solid with purity purity >99.0%

1H NMR (500 MHZ, DMSO-d6): δ ppm 10.19 (t, J=5.5 Hz, 1H), 4.61 (s, 2H), 4.20 (d, J=6.0 Hz, 2H), 3.68 (s, 3H), 2.26-2.24 (m, 4H), 1.79-1.76 (m, 4H), 1.67-1.60 (m, 6H), 1.39-1.23 (m, 4H), 1.14-1.09 (m, 2H). Mass: m/z: 406.1 [M−H]+, Theoretical exact mass: 407.2 (Mol Formula: C20H29N3O6).

PXRD (2θ): 6.1, 6.9, 7.5, 9.3, 9.5, 12.1, 14.8, 15.1, 17.2, 17.8, 19.1, 19.4, 20.0, 20.5, 21.6, 21.9, 22.6, 23.0, 23.7, 24.1, 24.5, 27.1, 27.2, 27.5, 27.9, 29.5, 30.1, 30.7 2θ.

### Method-2

In a reactor, glycine methyl ester hydrochloride (515 g, 4104 mmol) is added to a solution of 1,3-dicyclohexylpyrimidine-2,4,6 (1H,3H,5H)-trione (800 g, 2736 mmol), triethyl amine (554 g, 5472 mmol) and 1,1′-Carbonyldiimidazole (1.1 g, 6.8 mmol) in DCM (8 L) at 25-35° C. The solution is stirred at 25-35° C. for 1-2 hr. After completion of reaction, water (8 L) is added to the reaction mass and separated the layers. Organic layer is washed again with water (8 L) and concentrated under vacuum to obtain a solid precipitate. The obtained solid is slurried in hexane followed by methanol, then filtered and dried under vacuum to obtain N-[(1,3-dicyclohexylhexahydro-2,4,6-trioxo-5-pyrimidinyl)carbonyl]glycine methyl ester as off-white solid with purity >99.0%. 1H NMR (500 MHZ, DMSO-d6): δ ppm 10.18 (s, J=5.5 Hz, 1H), 4.64-4.59 (m, 2H), 4.20 (d, J=6.0 Hz, 2H), 3.68 (s, 3H), 2.29-2.22 (m, 4H), 1.77 (d, J=12.0 Hz, 4H), 1.62-1.57 (m, 6H), 1.30-1.22 (m, 4H), 1.14-1.09 (m, 2H). Mass: m/z: 408.3 [M+H]+, Theoretical exact mass: 407.2 (Mol Formula: C20H29N3O6).

PXRD (2θ): 4.6, 5.2, 5.7, 6.5, 7.0, 8.9, 9.3, 10.4, 11.2, 11.4, 13.9, 14.9, 15.5, 16.2, 16.8, 17.2, 17.7, 18.6, 19.3, 20.7, 21.2, 22.7, 23.3, 24.3, 25.0, 25.8, 27.1, 27.6 2θ.

PXRD pattern obtained is given in FIG. 25.

### Method-3

In a reactor, glycine methyl ester hydrochloride (3.22 g, 25.7 mmol) is added to a solution of 1,3-dicyclohexylpyrimidine-2,4,6 (1H,3H,5H)-trione (5 g, 17.10 mmol), triethyl amine (3.46 g, 34.2 mmol) and 1,1′-Carbonyldiimidazole (4.16 g, 25.7 mmol) in DMF (30 mL) at 25-35° C. The solution is stirred at 25-35 oC for 1-2 hr. After completion of reaction, methanol (30 mL), water (30 L), and acetone (30 mL) is added to the reaction mass and filtered the solid and dried under vacuum to obtain N-[(1,3-dicyclohexylhexahydro-2,4,6-trioxo-5-pyrimidinyl)carbonyl]glycine methyl ester as off-white solid with purity >98%.

PXRD (2θ): 4.3, 5.1, 6.0, 6.4, 7.0, 7.4, 8.7, 9.5, 10.3, 10.7, 14.9, 15.4, 16.4, 17.2, 17.4, 18.6, 20.6, 21.7, 21.9, 23.3, 24.5, 25.5, 25.6, 26.9, 27.6, 40.8 2θ.

### Method-4

In a reactor, glycine methyl ester hydrochloride (6.44 g, 51.3 mmol) is added to a solution of 1,3-dicyclohexylpyrimidine-2,4,6 (1H,3H,5H)-trione (10 g, 34.2 mmol), triethyl amine (3.46 g, 68.4 mmol) and 1,1′-Carbonyldiimidazole (4.16 g, 51.3 mmol) in ethyl acetate (100 mL) at 25-35° C. The solution is stirred at 25-35° C. for 1-2 hr. After completion of reaction, water (100 mL) is added to the reaction mass and separated the layers. Organic layer is washed again with water (100 mL) and concentrated under vacuum to obtain a solid precipitate. The obtained solid is slurried in hexane followed by methanol, then filtered and dried under vacuum to obtain N-[(1,3-dicyclohexylhexahydro-2,4,6-trioxo-5-pyrimidinyl)carbonyl]glycine methyl ester as off-white solid.

### Method-5

In a reactor, glycine methyl ester hydrochloride (6.44 g, 51.3 mmol) is added to a solution of 1,3-dicyclohexylpyrimidine-2,4,6 (1H,3H,5H)-trione (10 g, 34.2 mmol), triethyl amine (3.46 g, 68.4 mmol) and 1,1′-Carbonyldiimidazole (4.16 g, 51.3 mmol) in toluene (100 mL) at 25-35° C. The solution is stirred at 25-35° C. for 1-2 hr. After completion of reaction, water (100 mL) is added to the reaction mass and separated the layers. Organic layer is washed again with water (100 mL) and concentrated under vacuum to obtain a solid precipitate. The obtained solid is slurried in hexane followed methanol, then filtered and dried under vacuum to obtain N-[(1,3-dicyclohexylhexahydro-2,4,6-trioxo-5-pyrimidinyl)carbonyl]glycine methyl ester as off-white solid.

### Example-28: Preparation of 1,3-dicyclohexyl-2,4,6-trioxohexahydro pyrimidine-5-carbonyl)glycine (Daprodustat)

### Method-1

A stirred suspension of methyl (1,3-dicyclohexyl-2,4,6-trioxohexahydropyrimidine-5-carbonyl)glycinate (1.0 g, 2.46 mmol) in ethanol (10 mL) and water (10 mL) is treated with aq. sodium hydroxide (0.1 g, 2.46 mmol) solution and stirred at ambient temperature for 30 minutes. Then the reaction mixture is acidified with aq. hydrochloric acid (2.46 mmol) by the dropwise addition and stirred for 30-60 min. The solid is filtered and washed with water to afford the title compound as a white colour solid. 1H NMR (500 MHz, DMSO-d6): δ ppm 10.16 (t, J=5.5 Hz, 1H), 4.62 (t, J=10.0 Hz, 2H) 4.07 (d, J=5.0 Hz, 2H), 2.30-2.22 (m, 4H), 1.77 (d, J=13.0 Hz, 4H), 1.62-1.55 (m, 6H), 1.26 (q, J=13.0 Hz, 4H), 1.14-1.10 (q, J=10.0 Hz, 2H). Mass: m/z: 392.1 [M−H]+, Theoretical exact mass: 393.19 (Mol Formula: C19H27N3O6)

### Method-2

A stirred suspension of methyl (1,3-dicyclohexyl-2,4,6-trioxohexahydropyrimidine-5-carbonyl)glycinate (800 g, 1963 mmol) in methanol (8 L) is treated with 6N sodium hydroxide (1280 mL) solution and stirred at ambient temperature for 3-4 hrs. Then the reaction mixture is cooled to 15-20° C. and acidified with 6N hydrochloric acid (1520 mL) by addition. Then raised the reaction mass temperature to 25-35° C. and added water (5.2 L), stirred for 30-60 min. The obtained solid is filtered and slurried in water (16 L) to afford the title compound as a white colour solid with purity >99.0%. 1H NMR (500 MHZ, DMSO-d6): δ ppm 13.07 (bs, 1H), 10.18 (t, J=5.5 Hz, 4.0 Hz, 1H), 4.62 (bs, 2H) 4.11 (d, J=5.5 Hz, 2H), 2.26-2.24 (m, 4H), 1.77 (d, J=12.5 Hz, 4H), 1.63-1.60 (m, 6H), 1.26 (q, J=13.0 Hz, 4H), 1.28-1.23 (q, J=13.0 Hz, 4H), 1.12-1.09 (m, 2H). Mass: m/z: 391.9 [M−H]+, Theoretical exact mass: 393.19 (Mol Formula: C19H27N3O6).

### Example 29: Preparation of Crystalline Form DP-9 of Daprodustat

Daprodustat (100 g) is taken DMF (15V) in a reactor and contents are stirred. The temperature of the reaction mixture is raised to <500C and cooled. Clear solution is filtered and to the reaction mixture water (25 V) is added slowly. The precipitated solid is filtered, washed with water and dried. PXRD, DSC and TGA are given in FIG. 22, FIG. 23 and FIG. 24 respectively.

HPLC Purity: >99.5%, Total impurities: <0.5%, Unknown impurity: <0.15%

Below are the known impurities are

| Structure | Result |
|---|---|
| 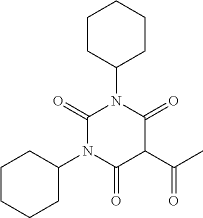 [SMILES: CC(=O)C1C(=O)N(C2CCCCC2)C(=O)N(C2CCCCC2)C1=O] | ND |
| 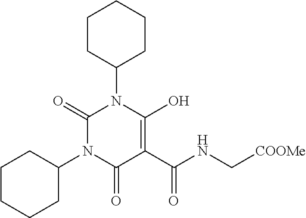 [SMILES: COC(=O)CNC(=O)c1c(O)n(C2CCCCC2)c(=O)n(C2CCCCC2)c1=O] | 0.08% |

Further Sieved the material through 30 mesh followed by micronized the material with 0.5 kg/cm2 feed and 0.5 kg/cm2 mill pressure to give particles having D90<20μ.

Although the present disclosure has been shown and described with reference to exemplary embodiments, those skilled in the art understand that, without departing from the spirit and scope of the present disclosure as defined by the appended claims and their equivalents, various modifications in form and detail may be made to the present disclosure. Therefore, the scope of the present disclosure should not be limited to the embodiments described herein, but should be determined not only by the appended claims, but also by the equivalents of the appended claims.

1. A crystalline form DP-9 of Daprodustat, wherein an X-ray powder diffraction pattern of the crystalline form DP-9 shows one or more characteristic peaks at 6.4, 7.2, 7.5, 12.3, 15.1, 15.5, 16.6, 18.5, 19.3, 21.0, and 27.6±0.2 2θ.

2. The crystalline form DP-9 of claim 1, wherein the X-ray powder diffraction pattern shows one or more characteristic peaks at 6.38, 7.18, 7.52, 10.78, 12.25, 12.92, 15.36, 16.65, 17.18, 18.06, 18.67, 19.14, 19.72, 20.84, 22.73, 24.78, 26.94, and 27.89.±0.2 2θ.

3. The crystalline form DP-9 of claim 1, further characterized by PXRD pattern as shown in FIG. 22.

4. The crystalline form DP-9 of claim 1, further characterized by DSC pattern as shown in FIG. 23.

5. The crystalline form DP-9 of claim 1, further characterized by TGA as shown in FIG. 24.

6. A process of preparing a crystalline form DP-9 of Daprodustat, the comprising:
dissolving Daprodustat in polar aprotic solvent to obtain a reaction mixture;
heating the reaction mixture to get a solution;
optionally adding another organic solvent;
isolating a solid obtained;
drying in a suitable temperature; and
optionally milling.

7. The process of claim 6, wherein the polar aprotic solvent is selected from N,N-dimethylformamide (DMF) and another organic solvent is selected from water.

8. A Pharmaceutical composition, comprising the crystalline form DP-9 of claim 1, and at least one pharmaceutically acceptable excipient.

9. A process for preparing Daprodustat, the process comprising,
converting compound 2 to compound 3 in presence of carbonyl reagent (C═O reagent) and amine reagent; and
converting compound 3 to Daprodustat.

10. A process for preparing Daprodustat, the method comprising,
converting compound 2 to compound 3 in presence of carbonyldiimidazole and Glycine methylester,
converting compound 3 to Daprodustat.
